(19) United States
(12) Patent Application Publication
(10) Pub. No.: US 2021/0142866 A1
(43) Pub. Date: May 13, 2021

(54) HYBRIDIZATION-BASED DNA INFORMATION STORAGE TO ALLOW RAPID AND PERMANENT ERASURE

(71) Applicant: William Marsh Rice University, Houston, TX (US)

(72) Inventors: David Yu ZHANG, Houston, TX (US); Alessandro PINTO, Houston, TX (US); Jangwon KIM, Houston, TX (US)

(73) Assignee: William Marsh Rice University, Houston, TX (US)

(21) Appl. No.: 17/057,620

(22) PCT Filed: May 23, 2019
(86) PCT No.: PCT/US2019/033754

§ 371 Date: Nov. 20, 2020

Related U.S. Application Data
(60) Provisional application No. 62/675,362, filed on May 23, 2018.

(51) Int. Cl. G16B 25/00; G16B 50/30; G06N 3/12

(52) U.S. Cl. CPC G16B 25/00; G06N 3/123; G16B 50/30

(57) ABSTRACT

Provided herein are methods for encoding information in DNA molecules in a way that allows rapid and permanent erasure of information. As such, methods of erasing such information are also provided. Also provided are compositions that so encode information.

### REFERENCE TO RELATED APPLICATIONS

The present application claims the priority benefit of U.S. provisional application No. 62/675,362, filed May 23, 2018, the entire contents of which is incorporated herein by reference.

### STATEMENT REGARDING FEDERALLY SPONSORED RESEARCH

This invention was made with government support under Grant No. R01 HG008752 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on May 15, 2019, is named RICEP0045WO_ST25.txt and is 6.1 kilobytes in size.

### BACKGROUND

### 1. Field

Provided herein are methods to encode, copy, erase, and decode information in DNA molecules. Also provided are compositions comprising DNA molecules whose sequences encode such information.

### 2. Description of Related Art

As modern data storage demands increase at an exponential pace, new high-density information storage media are needed as conventional silicon-based materials reach the quantum mechanical limits of fabrication. Additionally, highly important information that must be reliably archived for long-term storage and retrieval require robust storage methods that do not require routine copying to preserve information integrity; for example, tape information storage must be “rewritten” every 10 years.

Information storage in DNA molecules is an emerging solution to both of the above demands: DNA is highly information dense, and also has an extremely long chemical half-life—over 500 years by some estimates. Additionally, recent advances in both high-throughput DNA synthesis and DNA sequencing suggest that DNA may be economically competitive with other information storage media in the 5-10 year time horizon. For these reasons, a number of recent publications have described and demonstrated proof-of-concept experiments demonstrating information storage with DNA.

Data privacy and security is of increasing concern in today world, with sensitive data spanning patient medical histories to confidential corporate documents to government and military secrets. To facilitate proper protection of classified information, information stored on a medium must be rapidly and permanently erasable. However, all common data storage methods today are difficult to permanently erase. For example, degaussing or physically destroying hard drives is frequently incomplete, and information can still be recovered by dedicated effort. Information encoded DNA sequence likewise can be in principle be erased by bleach or acid treatment, but may require long reaction times and rigorous mixing to ensure complete destruction of information. As such, methods for encoding information in DNA that allow rapid and permanent erasure are needed.

### SUMMARY

Provided herein are methods to encode, copy, erase, and decode information in DNA molecules. Unlike standard information storage methods for computer files (e.g., solid-state hard drives, tape) and other DNA-based information storage methods, the described methods allow rapid and permanent erasure of information. This is expected to be of significant value for highly sensitive or confidential information, including military documents, classified court records, and HIPAA-protected patient medical records.

In one embodiment, composition are provided comprising a population of DNA molecules, wherein the population comprises true information DNA molecules, false obfuscation DNA molecules, and truth marker DNA oligonucleotides, wherein the true information DNA molecules and the false obfuscation DNA molecules each comprise a first sequence that is complementary to a portion of a sequence of the truth marker DNA oligonucleotides, wherein the first sequence of the true information DNA molecules is hybridized to the truth marker DNA oligonucleotides, wherein the first sequence of the false obfuscation DNA molecules is not hybridized to the truth marker DNA oligonucleotides, wherein the true information DNA molecules and the false obfuscation DNA molecules each comprise an address region, wherein the address region of each true information DNA molecule is unique among the true information DNA molecules in the population, wherein the address region of each false information DNA molecule is unique among the false information DNA molecules in the population, wherein one true information DNA molecule and at least one false information DNA molecule in the population share an identical address region.

In some aspects, the first sequence of the false obfuscation DNA molecules is single stranded. In some aspects, the population further comprises false marker DNA oligonucleotides. In certain aspects, a portion of the false marker DNA oligonucleotides is at least partially complementary to the first sequence of both the true information DNA molecules and the false obfuscation DNA molecules. In certain aspects, the false marker DNA oligonucleotides and the truth marker DNA oligonucleotides comprise different sequences. In certain aspects, the false marker DNA oligonucleotides comprise a chemical functionalization. In certain aspects, the first sequence of the false obfuscation DNA molecules is hybridized to the false marker DNA oligonucleotides. In certain aspects, the false marker DNA oligonucleotides comprise a 3′ functionalization that prevents extension by a DNA polymerase. In certain aspects, the first sequence is between 10 and 50 nucleotides long. In certain aspects, the true information DNA molecules and the false obfuscation DNA molecules are each, independently, between 50 and 2000 nucleotides long. In certain aspects, the first regions of the true information DNA molecules are located towards the 5′ end of the true information DNA molecules. In certain aspects, the truth marker DNA oligonucleotides comprise a primer binding region that is not complementary to the true information DNA molecules.

In one embodiment, methods are provided of encoding an information-bearing or obfuscation file in DNA molecules, the methods comprising: (a) obtaining an input file in ASCII/hexadecimal format; (b) independently translating each ASCII character/byte from 00 to FF in hexadecimal to a five nucleotide DNA sequence; (c) dividing the concatenated DNA sequence representing the entire input file into a set of message sequences; (d) providing and encoding in DNA a unique address sequence identifying the position within the DNA sequence for each message sequence; (e) designing a truth marker binding region sequence; (f) constructing information DNA molecule sequences by concatenating from 5′ to 3′ the truth marker binding region sequence, the unique address sequences, and corresponding message sequences; and (g) chemically synthesizing information DNA molecules comprising the information DNA molecule sequences.

In some aspects, the information DNA molecules further comprises one or more primer binding regions located on the 5′ and/or 3′ end of the information DNA molecule sequence. In some aspects, the obfuscation DNA molecules further comprises one or more primer binding regions located on the 5′ and/or 3′ end of the information DNA molecule sequence. In some aspects, each ASCII character/byte is converted to one 2-bit region and two 3-bit regions, wherein the 2-bit region is mapped to G, C, A, or T, and wherein the 3-bit regions are each mapped to CA, CT, GA, GT, TC, TG, AC, or AG.

In one embodiment, provided herein are populations of information DNA molecules made by the methods of any one of the present embodiments.

In one embodiment, methods are provided for preparing a DNA solution encoding information that is amenable to rapid erasure, the method comprising: (a) preparing a solution of information DNA molecules encoding an information-bearing file according to the method of any one of the present embodiments; (b) hybridizing the solution of information DNA molecules to a solution of truth marker DNA oligonucleotide molecules; (c) preparing at least one solution of obfuscation DNA molecules encoding an obfuscation file according to the method of any one of the present embodiments; and (d) combining the hybridizes solution of part (b) with the at least one solution of obfuscation DNA molecules of part (c).

In some aspects, the methods further comprise hybridizing the at least one solution of obfuscation DNA molecules to a solution of false marker DNA oligonucleotide molecules prior to combining in part (d). In some aspects, the truth marker DNA oligonucleotides are present at a molar quantity that is smaller than or equal to the molar quantity of information DNA molecules. In some aspects, the false marker DNA oligonucleotides are present at a molar quantity that is greater than or equal to the molar quantity of obfuscation DNA molecules. In some aspects, the hybridizing of part (b) comprises heating the combined solutions to at least 70° C. and then cooling the combined solutions to 50° C. or lower. In some aspects, hybridizing the at least one solution of obfuscation DNA molecules to a solution of false marker DNA oligonucleotide molecules prior to combining in part (d) comprises heating the combined solutions to at least 70° C. and then cooling the combined solutions to 50° C. or lower.

In one embodiment, provided are DNA solutions encoding information that is amenable to rapid erasure made by the method of any one of the present embodiments.

In one embodiment, provided are methods of erasing information encoded in a DNA solution of any one of the present embodiments, the method comprising heating the DNA solution an elevated temperature for a duration of no less than 15 seconds. In some aspects, the elevated temperature is approximately 50° C., 55° C., 60° C., 65° C., 70° C., 75° C., 80° C., 85° C., 90° C., 95° C., or 100° C. In some aspects, the duration of the heating is approximately 15 seconds, 30 seconds, 45 seconds, 1 minute, 2 minutes, 3 minutes, 5 minutes, 10 minutes, 15 minutes, 20 minutes, 30 minutes, or 60 minutes.

In one embodiment, provided are methods of reading information encoded in a DNA solution of any one of the present embodiments, the method comprising: (a) adding a DNA polymerase, dNTPs, and buffers to the solution; (b) incubating the mixture of part (a) at a temperature amenable to enzymatic extension of the truth marker based on the hybridized information DNA molecules; (c) preparing a next-generation sequencing (NGS) library based on the polymerase-extended truth markers of part (b); (d) performing NGS; (e) analyzing NGS reads to determine the dominant message sequence for each address sequence; and (f) reassembling the information-bearing file from the dominant message sequence for each address sequence.

In some aspects, the preparation of the NGS library based on polymerase-extended truth markers comprises ligation of sequencing adaptors to double-stranded DNA molecules. In some aspects, the NGS library preparation further comprises polymerase chain reaction (PCR) amplification using sequencing adaptors. In some aspects, the preparation of the NGS library based on polymerase-extended truth markers comprises polymerase chain reaction (PCR) amplification comprising a primer that includes a sequencing adaptor at or near the 5′ region and a sequence specific to the truth marker DNA oligonucleotide but not to the false marker DNA oligonucleotide. In some aspects, the NGS library preparation further comprises appending sample indexes using PCR.

In one embodiment, provided are methods of erasing information encoded in a DNA solution of any one of the present embodiments, the method comprising exposing the DNA solution to a temperature above room temperature for a duration of no less than the estimated half-life of the duplex comprising the truth marker and the first sequence. In some aspects, the half-life is calculated as

$${t}_{1 / 2} = \frac{{e}^{{ΔG}^{\underline{o}} / RT}}{{k}_{f}}$$

where t_{1/2} is half-life, R is the gas constant, T is the exposure temperature, ΔG° is the Gibbs free hybridization of a duplex, and k_{f} (=10⁶ M·⁻¹ s⁻¹) is the rate constant of hybridization.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Modulating information duration via temperature using hybridization-based DNA encoding. (FIG. 1A) Illustration of information DNA molecules bearing true messages and obfuscation DNA molecules bearing false messages. The information DNA molecules have a “truth marker” oligonucleotide hybridized to the truth marker binding site. The obfuscation DNA molecules either do not have any oligonucleotides hybridized to the truth marker binding site, or have a “false marker” oligonucleotide hybridized to the truth marker binding site. The false marker is distinct from the truth marker in chemical identity; for example, the X shown at the 3′ end of the false marker may be a 3-carbon spacer or an inverted nucleotide that prevents polymerase extension. (FIG. 1B) Implementation of hybridization-based DNA encoding. Messages intended to be part of the communicated information are pre-hybridized to truth markers, DNA oligonucleotides with an extensible 3′ end and a 5′ overhang sequence. Confounding noise molecules corresponding to nonsensical information is pre-hybridized to false markers, DNA oligonucleotides with blocked 3′ ends and lacking the 5′ overhang sequence. The sequences of the false marker and truth marker where they bind their DNA target are the same, so any message or noise molecule can bind with roughly equal favorability to either the truth marker or the false marker. The messages and noise are mixed in the DNA solution. Upon heating, the hybridization of the truth markers to the intended messages is disrupted. Subsequent cooling to room temperature would result in a random association of truth markers to messages and noise, and the information regarding which molecules correspond to messages vs. noise is permanently lost (see FIG. 4A).

FIG. 2. The half-life of truth marker hybridization is strongly temperature-dependent. Plotted here is the calculated half-life of a 20 nt truth marker with the given sequence (SEQ ID NO. 21) at different temperatures, based on the two-state model of DNA binding and published DNA thermodynamics parameters, and assuming a hybridization rate constant of kf=10\hat{ }6/M/s. Half-life values are calculated based on kr=kf/Keq, where Keq=e\hat{ }(−ΔG°/RT), with ΔG° being the computed standard free energy of hybridization of the sequence with its complement in 0.15 M Na+(evaluated using the Nupack DNA folding software), R being the universal gas constant, and T being the temperature in Kelvin.

FIG. 3. Experimental characterization of truth marker binding kinetics via polyacrylamide gel electrophoresis. Demonstration of message erasure through polyacrylamide gel electrophoresis. Three gel images are the same gel scanned in three different fluorescence filter sets. Lanes 1 and 2 are references showing the unhybridized intended message (i.e., true message) and the noise DNA (i.e., false message), respectively. Lanes 3 and 4 show the intended message pre-hybridized to the truth marker and the noise DNA pre-hybridized to the false marker, respectively. Lanes 5 and 6 are noise DNA pre-hybridized to FAM-attached truth marker and intended message pre-hybridized to ROX-attached false marker. Lane 7-11 shows the mixture of the species in Lanes 3 and 4 incubated for different amounts of time at different temperatures. After 1 hour and 1 week at 25° C. (Lanes 7 and 8, respectively), the truth marker and false markers remain hybridized to their initially bound DNA molecules, showing truth markers attached to intended messages and false markers attached to noise. However, heating the mixture at 60° C. or 95° C. shows redistribution of truth marker and false marker to either the intended message or the noise, so that the intended message loses its truthness.

FIGS. 4A-B. Rapid and permanent erasure of information encoded in a solution of information DNA molecules and obfuscation DNA molecules. Upon heating to a temperature that is higher than the storage temperature for an extended period of time sufficient to melt duplex DNA species in solution (FIG. 2; FIG. 4B), the truth marker dissociates from information DNA molecules, and information regarding which messages are truth and which messages are false are permanently erased. After cooling, the truth markers randomly bind to either information DNA molecules or obfuscation DNA molecules (FIG. 4A).

FIG. 5. Example of information and obfuscation DNA molecule structure. In this example, the truth marker comprises region 6 at its 5′ end, which is later used as a forward primer binding site for downstream PCR. Region 1 of the truth marker is complementary to region 2, the truth marker binding site. The false marker comprises region 1 and a 3-carbon functionalization at the 3′ end to prevent extension. Each information and obfuscation DNA molecule has an address sequence, a message sequence, and a reverse primer binding region. To enable rapid information erasure, each unique address should have one corresponding information DNA molecule and at least one corresponding obfuscation DNA molecule.

FIG. 6. Information encoding scheme. Information files used by computer systems are typically stored in ASCII format, with each byte taking on a value between 0 and 255 (00 to FF in hexadecimal). For example, the lowercase letter “o” is 6F in hexadecimal in ASCII format, which in binary is represented as “01101111.” The 8 bits are then grouped into 1 group of 2 bits, and 2 groups of 3 bits, and the mapping table listed to the lower-left is used to convert the letter “o” into the DNA sequence “TCTGT.”

FIG. 7. Method for reading out messages encoded in information DNA molecules from a non-erased mixture of information DNA molecules and obfuscation DNA molecules. Truth markers are extended by DNA polymerase and the messages encoded in DNA information molecules are copied. Only the extended truth molecules are able to be PCR amplified in the subsequent step.

FIGS. 8A-B. Graphical display of data obtained reading a non-erased solution of information DNA molecules and obfuscation DNA molecules. (FIG. 8A) Here, three sets of obfuscation DNA molecules (corresponding to three different images) were used in conjunction with one set of information DNA molecules. The left-most image is the intended message, the middle image is the read message, and the right image is the read message after erasure (15 minutes at 95° C.). The gray pixels in the middle and right images indicate addresses in which a message was not recovered, either due to oligonucleotide synthesis non-uniformity or NGS non-uniformity. The images and information DNA molecules include 24-bit color encoded in RGB format. (FIG. 8B) Desired information, in this case a bitmap image, is encoded as a DNA solution. The information can be stored stably for extended periods of time at room temperature or lower, but is quickly and permanently erased upon exposure to elevated temperatures (e.g. 95° C.).

FIG. 9. Schematic for preparing DNA oligonucleotides as information DNA molecules or obfuscation DNA molecules from a mixed DNA synthesis pool. The pool is a mixture of several “files” where each file has its unique file primer binding region. One of the files is amplified with a phosphate-modified file forward primer and a unique phosphorothioate-modified file reverse primer. Lambda exonuclease is used to treat the file to remove phosphate-modified oligos. Subsequently, to convert the file amplicons into information DNA molecules, truth marker oligonucleotides are added. Optionally, to convert the file amplicons into obfuscation DNA molecules, false marker oligonucleotides are added.

FIGS. 10A-H. Encoding ASCII files as DNA. (FIG. 10A) Each byte is encoded as a word of 5 DNA nucleotides. The mapping is 80% efficient compared to the minimum 4 nt needed to encode 256 possible characters. (FIG. 10B) Mapping table. Importantly, this mapping restricts G/C content of DNA sequences to between 40% and 60%, and guarantees that there are no homopolymer stretches of more than 3 nt. (FIG. 10C) Each DNA oligonucleotide used for information storage can be abstracted as 4 domains. The B region is a sequence common to all oligos, in which the truth marker and false marker can bind. The A region corresponds to the address of the message, relative to a file position. The M region corresponds to the message content. The L region corresponds to a library-specific primer sequence used for pre-amplification from chip-synthesized oligo pools; the L region is removed in the final oligos used for storage. (FIG. 10D) Bitmap images of 8 pieces of artwork are here encoded as DNA. Displayed here are the reconstituted images based on the designed oligo pool synthesized by Twist Biosciences, read via NGS on an Illumina MiSeq. (FIG. 10E) Distribution of NGS reads mapped to the library mapped to “The Bull”. 16.11% of reads discarded from further analysis, because they did not exhibit the expected DNA oligo format, either due to oligo synthesis error or due to sequencing error. (FIG. 10F) Spatial distribution of sequencing depth. Each DNA oligo corresponds to a non-overlapping block of 2×2 pixels. (FIG. 10G) Fraction of NGS reads mapping to each pixel block with the exact expected sequence, based on position (left) and sorted by rank (right). (FIG. 10H) The fraction of NGS reads corresponding to the plurality of each pixel block. Note that a small fraction of pixel blocks converge to an incorrect set of pixel information.

FIGS. 11A-F. Information storage and reading. (FIG. 11A) Reading images encoded in DNA, using a mixture of 1 message file and 1 noise file. Top image corresponds to the message file (pre-hybridized to truth marker) and bottom image corresponds to the noise file (pre-hybridized to false marker). Middle image corresponds to the recovered image after erasing the message by heating for 15 minutes at 95° C. (FIG. 11B) Spatial distribution of missing pixels (black) and incorrect pixels corresponding to noise (gray). The vertical gray stripe in the top image is expected because the first image has no encoded information there. (FIG. 11C) Distribution of NGS reads across all pixels. (FIG. 11D) Distribution of the number of NGS reads matching perfectly in each pixel block. In the second image, the “matched reads” correspond to the first image. (FIG. 11E) Fraction of NGS reads mapped to each pixel block exactly matching the expected DNA message. (FIG. 11F) Fraction of each pixel block mapping to the highest frequency NGS read in each block (plurality).

FIGS. 12A-B. Information storage and reading from a mixture of 8 images. (FIG. 12A) Read images after incubating image mixture at 25° C. for 1 week. (FIG. 12B) Read images after incubating image mixture at 95° C. for 15 minutes.

FIGS. 13A-J. Quality of Chip-synthesized oligo pools. (FIG. 13A) 8 Images shown here are the retrieved images of designed oligo pool. Missing pixels are labeled with gray block in each image. Oligos whose correct reads are less than 5 are regarded as poorly synthesized oligos and re-ordered as second oligo pool, to fill the missing pixels. (FIG. 13B) Pie graph describing the fraction of perfectly synthesized oligo pools. We only used the perfectly synthesized oligos for further analysis. (FIG. 13C) Spatial distribution and histogram of sequencing depth. In the histogram, oligos having less than 5 exact hits are described. This oligos are re-ordered as the second pool. (FIG. 13D) Ratio of exact NGS reads mapping to each pixel block. (FIG. 13E) Plurality ratio, the number of dominant reads divided by the number of total reads, mapping to each pixel block. (FIG. 13F) 8 images retrieved from second pool spiked-in oligo pool. Missing pixels are labelled with gray block in each image, but missing pixels are hard to find in almost all images after second pool is added. (FIG. 13G) Pie graph describing the fraction of perfectly synthesized oligo pools. (FIG. 13H) Spatial distribution and histogram of sequencing depth. (FIG. 13I) Ratio of exact NGS reads mapping to each pixel block, which is increased overall after second pool is spiked-in. (FIG. 13J) Plurality ratio, the number of dominant reads divided by the number of total reads, mapping to each pixel block.

FIGS. 14A-F. Information storage and reading. (FIG. 14A) Decoded images encoded in DNA, using a mixture of 1 message file and 7 noise files. The message file was pre-hybridized to truth marker and noise files were pre-hybridized to false marker respectively. Size of image was set as 240×320 upon decoding. (FIG. 14B) Spatial distribution of missing pixels (black) and incorrect pixels corresponding to noise (gray). Outer parts of original image of the message file in 240×320 domain were displayed in gray. (FIG. 14C) Pie chart showing the distribution of NGS reads. The fraction of NGS reads match exactly to the original message file, the NGS reads match exactly to the original noise file, the ratio of NGS reads containing error in either address part or message part, and the ratio of NGS reads whose length is different from originally synthesized oligos are shown. (FIG. 14D) Distribution of the number of exact NGS reads across all pixels. (FIG. 14E) Mapping the ratio of exact NGS reads mapping to each pixel. (FIG. 14F) Plurality ratio in each block which corresponds to the fraction of each pixel block mapping to the number of dominant NGS reads.

FIGS. 15A-F. Information storage and reading, showing information decay after 1 week. (FIG. 15A) Reading images encoded in DNA, using a mixture of 1 message file and 7 noise files. Unlike FIGS. 14A-F, the mixture was incubated for 1 week at room temperature to test information decay, and then moved on to the next procedure for decoding/reading. Size of image was set as 240×320 upon decoding. (FIG. 15B) Spatial distribution of missing pixels (black) and incorrect pixels corresponding to noise (gray). Outer parts of original image of the message file in 240×320 domain were displayed in gray. (FIG. 15C) Pie chart showing the distribution of NGS reads. The fraction of NGS reads match exactly to the original message file, the NGS reads match exactly to the original noise file, the ratio of NGS reads containing error in either address part or message part, and the ratio of NGS reads whose length is different from originally synthesized oligos are shown. Even after 1 week of incubation, the results hardly indicate information decay. (FIG. 15D) Distribution of the number of exact NGS reads across all pixels. (FIG. 15E) Mapping the ratio of exact NGS reads mapping to each pixel. (FIG. 15F) Plurality ratio in each block which corresponds to the fraction of each pixel block mapping to the number of dominant NGS reads.

FIGS. 16A-F. Information erasure through heating the mixture at 95° C. (FIG. 16A) Reading images encoded in DNA, after erasing information in a mixture of 1 message file and 7 noise files. All 8 images look alike and hard to recognize the original image. Size of image was set as 240×320 upon decoding. (FIG. 16B) Spatial distribution of missing pixels (black) and incorrect pixels corresponding to noise (gray). Outer parts of original image of the message file in 240×320 domain were displayed in gray. After erasure, the majority of pixels correspond to noise. (FIG. 16C) Pie chart showing the distribution of NGS reads. The fraction of NGS reads match exactly to the original message file, the NGS reads match exactly to the original noise file, the ratio of NGS reads containing error in either address part or message part, and the ratio of NGS reads whose length is different from originally synthesized oligos are shown. After erasure, the perfect true message become dominant while the perfect noise/false message is decreased. (FIG. 16D) Distribution of the number of exact NGS reads across all pixels. Although all 8 reading images look the same after erasure, some of the graphs have the patterns of the original images. It is because this graph is the result of matching the reading image to the original image. (FIG. 16E) Mapping the ratio of exact NGS reads mapping to each pixel. (FIG. 16F) Plurality ratio in each block which corresponds to the fraction of each pixel block mapping to the number of dominant NGS reads.

FIGS. 17A-F. Incomplete information erasure through heating the mixture at 60° C. (FIG. 17A) Reading images encoded in DNA, after erasing information in a mixture of 1 message file and 7 noise files. Even with erasure at 60° C., original information (image) can be hardly recognized. Size of image was set as 240×320 upon decoding. (FIG. 17B) Spatial distribution of missing pixels (black) and incorrect pixels corresponding to noise (gray). Outer parts of original image of the message file in 240×320 domain were displayed in gray. After erasure, the majority of pixels correspond to noise. (FIG. 17C) Pie chart showing the distribution of NGS reads. The fraction of NGS reads match exactly to the original message file, the NGS reads match exactly to the original noise file, the ratio of NGS reads containing error in either address part or message part, and the ratio of NGS reads whose length is different from originally synthesized oligos are shown. Compared to the file erased at 95° C., this file has slightly larger perfect true message region and smaller perfect noise/false message region. (FIG. 17D) Distribution of the number of exact NGS reads across all pixels. Although all 8 reading images look the same after erasure, some of the graphs have the patterns of the original images. It is because this graph is the result of matching the reading image to the original image. (FIG. 17E) Mapping the ratio of exact NGS reads mapping to each pixel. (FIG. 17F) Plurality ratio in each block which corresponds to the fraction of each pixel block mapping to the number of dominant NGS reads. In the histogram, the plurality ratio is distributed in a higher region than that of file erased at 95° C.

FIG. 18. Bar graph showing the ratio of correct, missing, and incorrect pixels of reading images. Ratios are the average values of 8 images. For original Twist pool, mixture of a message file and noise files, and the mixture incubated for 1 week at room temperature (Lanes 1-3) shows dominant ratio of correct pixels. On the other hand, in erased files (Lanes 4-6), incorrect or missing pixels are much dominant Lanes 5 and 6 are the graph analyzed with the reads whose plurality ratios are over 0.5. Truth markers and false markers are more distributed at 95° C. than 60° C., showing more missing pixels in files erased at 95° C. Lane 1: Original Twist pool. Lane 2: Mixture of a message file and noise file. Lane 3: Mixture of a message file and noise files stored for 1 week at RT. Lane 3: Mixture of a message file and noise files erased at 95° C. Lane 4: Mixture of a message file and noise files erased at 95° C. Lane 5: Mixture of a message file and noise files erased at 95° C. (Cutoff: plurality ratio >0.5). Lane 6: Mixture of a message file and noise files erased at 60° C. (Cutoff: plurality ratio >0.5).

### DETAILED DESCRIPTION

Encoding information in DNA is an emerging area with significant investment. Compared to traditional media for information storage, DNA holds the potential to have significantly higher information density and longer storage lifetimes. However, current methods to encode information in DNA are extremely difficult to permanently erase, making the approach less suitable for highly sensitive information.

The methods provided herein use the strong temperature dependence of DNA hybridization half-lives to encode information in a way that can be easily erased or obfuscated via a simple and rapid heating procedure. In brief, DNA molecules corresponding to true messages (i.e., “true information DNA molecules”) are pre-hybridized to “truth marker DNA oligonucleotides,” and then mixed with DNA molecules corresponding to false messages (i.e., “false obfuscation DNA molecules”). Upon heating, the truth markers are dissociated from the true messages, and will randomly hybridize with DNA molecules corresponding to true or false messages after cooling.

The basis of the rapid erasure aspect of the present invention is that it is exponentially difficult to reconstruct a message from multiple components when there are multiple possible options for each component. For example, if there are N=10,000 components and M=2 options for each component of which only one option is correct, then there are 2\hat{ }10000≈10\hat{ }3000 possible messages, and it is practically impossible to find the one true message out of all the possible messages. Thus, DNA information storage can be implemented via a set of true messages (information) and at least one set of false messages (obfuscation).

The information in the true messages can be encoded into DNA sequences in a variety of means. One example of an encoding strategy for translating ASCII files into DNA sequences is shown in FIG. 6. Information files used by computer systems are typically stored in ASCII format, with each byte taking on a value between 0 and 255 (00 to FF in hexadecimal). For example, the lowercase letter “o” is 6F in hexadecimal in ASCII format, which in binary is represented as the following 8 bits: “01101111.” The 8 bits can then grouped into 1 group of 2 bits and 2 groups of 3 bits (i.e., 01 101 111), and the mapping table shown in the lower-left of FIG. 6 can be used to convert the letter “o” into the DNA sequence “TCTGT.” As such, each byte is translated into a 5 nucleotide DNA sequence in a 1-to-1 mapping. Consequently, this mapping is 80% efficient (every 8 bits is converted into 5 nucleotides that each contain 2 bits of information). One advantage of this encoding method is that all sequences thus generated have G/C contents between 40% and 60%, making such sequences amenable to reliable synthesis and sequencing. Another advantage of this encoding method is that no sequence thus generated will have a continuous homopolymer stretch of more than three nucleotides, avoiding undesirable DNA secondary and tertiary structures such as G-quadruplexes. Another advantage of this encoding method is that the DNA sequence format allows easy detection of DNA synthesis side products that include internal deletions.

Once the information has been encoded into a DNA sequence, the DNA sequence can be fragmented into DNA-encoded true messages. Each message may be between about 50 and about 2000 nucleotides in length, or any length derivable therein. For example, a message may be about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, or about 2000 nucleotides long. Each message can be associated with an address that identifies the location of the encoded message within the DNA sequence so that the DNA sequence can be reconstructed based on the messages. The address may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides long. In order for the DNA sequence to encode erasable information, the population of DNA-encoded messages is kept in solution with false DNA messages. Each address associated with a true message is also present on a second DNA molecules where it is associated with a false message. As such, once the truth marker is lost (i.e., dehybridized) from the true DNA message, there is no way to identify which message with a specific address is the true DNA message.

In the readable form of a DNA-encoded message, there is a “truth marker oligonucleotide” that is bound to all information DNA molecules that bear true messages (i.e., “true information DNA molecules”) (FIG. 1A). The truth markers have an extensible 3′ end and a 5′ overhang sequence. The “false obfuscation DNA molecules” that bear false messages also have a truth marker binding site that allows a truth marker to be bound, but are not initially bound to truth markers. Alternatively, the false obfuscation DNA molecules may have a “false marker” oligonucleotide hybridized to the truth marker binding site. The false marker is distinct from the truth marker in chemical identity; for example, the X shown at the 3′ end of the false marker may be a 3-carbon spacer or an inverted nucleotide that prevents polymerase extension. The false markers may also lack the 5′ overhang sequence. The sequences of the false marker and truth marker where they bind their DNA target are the same, so any message or noise molecule can bind with roughly equal favorability to either the truth marker or the false marker. The messages and noise are mixed in the DNA solution. Upon heating, the hybridization of the truth markers to the intended messages is disrupted. Subsequent cooling to room temperature would result in a random association of truth markers to messages and noise, and the information regarding which molecules correspond to messages vs. noise is permanently lost.

The methods provided leverage the strong temperature dependence of the half-life of DNA hybridization interactions (FIG. 2). Upon heating to a temperature that is at least the melting temperature of the truth marker, the truth marker dissociates from the true messages (FIGS. 3,4A,4B), and it becomes impossible to distinguish the original information DNA molecules from the original obfuscation DNA molecules. Even cooling the heated solution down to room temperature will not restore the information, as the truth marker will then randomly associate with true and false messages. In contrast, when the original DNA-encoded message is kept at room temperature or a suitably cold temperature, the half-life of truth marker dissociation is extremely long, allowing the messages to be preserved long-term in the absence of willful information destruction. The temperature-dependent half-life of the encoded information can also be seen as a method for producing “self-destroying” messages that are intended to be viewed within a limited time after production.

Obfuscation DNA molecules that bear false messages may be hybridized to a “false marker oligonucleotide” at the truth marker binding region (see FIG. 1B). This false marker is distinct in identity from the truth marker, either in DNA sequence or in chemical modification. As shown in FIG. 5, the truth marker may have an additional 5′ sequence (region 6) that is used as the forward primer binding site for downstream PCR amplification, and is not modified at the 3′ end. In contrast, the false marker might not have the 5′ forward primer binding region, and is functionalized at the 3′ end to prevent DNA polymerase extension. Such functionalization may be a 3-carbon spacer. As such, the false obfuscation DNA molecules and the true information DNA molecules are otherwise similar in structure: they each comprise an address sequence, a message sequence, and a reverse primer binding region. To enable rapid information erasure, each unique address should have one corresponding true information DNA molecule and at least one corresponding false obfuscation DNA molecule.

One example, as described in detail in Example 1, of the information reading process for a non-erased message is illustrated in FIG. 7. DNA polymerase will extend the truth marker, copying the true message from the information DNA molecule. Only the extended truth marker has both a forward primer binding site and a reverse primer binding site, and can be subsequently amplified by PCR. The PCR primers used also include sequencing adapters at the 5′ end to allow subsequent NGS analysis to read out the messages encoded in the information DNA molecules. FIGS. 8A-B show the results of reading a non-erased DNA solution and an erased DNA solution for comparison. Desired information, in this case a bitmap image, is encoded as a DNA solution. The information can be stored stably for extended periods of time at room temperature or lower, but is quickly and permanently erased upon exposure to elevated temperatures (e.g. 95° C.). FIG. 9 shows how information DNA molecules and obfuscation DNA molecules can be prepared from a larger synthesis pool of many thousands to millions of oligonucleotide species. The pool is a mixture of several “files” where each file has its unique file primer binding region. One of the files is amplified with a phosphate-modified file forward primer and a unique phosphorothioate-modified file reverse primer. Lambda exonuclease is used to treat the file to remove phosphate-modified oligos. Subsequently, to convert the file amplicons into information DNA molecules, truth marker oligonucleotides are added. Optionally, to convert the file amplicons into obfuscation DNA molecules, false marker oligonucleotides are added.

Another example of the encoding strategy for translating ASCII files into DNA sequences is shown in FIGS. 10A-H. Here again, each byte is encoded as a word of 5 DNA nucleotides (FIG. 10A). The mapping is 80% efficient compared to the minimum 4 nt needed to encode 256 possible characters. Importantly, this mapping restricts G/C content of DNA sequences to between 40% and 60%, and guarantees that there are no homopolymer stretches of more than 3 nt (FIG. 10B). Each DNA oligonucleotide used for information storage can be abstracted as 4 domains (FIG. 10C). The B region is a sequence common to all oligos, in which the truth marker and false marker can bind. The A region corresponds to the address of the message, relative to a file position. The M region corresponds to the message content. The L region corresponds to a library-specific primer sequence used for pre-amplification from chip-synthesized oligo pools; the L region is removed in the final oligos used for storage.

Bitmap images of 8 pieces of artwork are here encoded as DNA (FIG. 10D). Displayed are the reconstituted images based on the designed oligo pool synthesized by Twist Biosciences, read via NGS on an Illumina MiSeq. The distribution of NGS reads mapped to the library mapped to “The Bull” are shown here as a specific example (FIG. 10E). 16.11% of reads discarded from further analysis, because they did not exhibit the expected DNA oligo format, either due to oligo synthesis error or due to sequencing error. The spatial distribution of sequencing depth is shown in FIG. 10F. Each DNA oligo corresponds to a non-overlapping block of 2×2 pixels. The fraction of NGS reads mapping to each pixel block with the exact expected sequence, based on position (left) and sorted by rank (right) is shown in FIG. 10G. The fraction of NGS reads corresponding to the plurality of each pixel block is shown in FIG. 10H. Note that a small fraction of pixel blocks converge to an incorrect set of pixel information.

The quality of chip-synthesized oligo pools were assessed in FIGS. 13A-J. First, the 8 images shown in FIG. 13A are the retrieved images of a designed oligo pool. Missing pixels are labeled with a block in each image. Oligos whose correct reads were less than 5 were regarded as poorly synthesized oligos and re-ordered as second oligo pool, to fill the missing pixels. FIG. 13B provides a pie graph describing the fraction of perfectly synthesized oligo pools. Only the perfectly synthesized oligos were used for further analysis. The spatial distribution and histogram of sequencing depth are shown in FIG. 13C. In the histogram, oligos having less than 5 exact hits are labeled. These oligos were re-ordered as the second pool. The ratio of exact NGS reads mapping to each pixel block is shown in FIG. 13D. The plurality ratio, i.e., the number of dominant reads divided by the number of total reads, mapping to each pixel block is shown in FIG. 13E. Next, the 8 images retrieved from the second-pool-spiked-in oligo pool are shown in FIG. 13F. Missing pixels are labeled with a block in each image, but missing pixels are hard to find in almost all images after the second pool is added. FIG. 13G provides a pie graph describing the fraction of perfectly synthesized oligo pools. The spatial distribution and histogram of sequencing depth are shown in FIG. 13H. The ratio of exact NGS reads mapping to each pixel block, which is increased overall after second pool is spiked-in, is shown in FIG. 13I. The plurality ratio, the number of dominant reads divided by the number of total reads, mapping to each pixel block is shown in FIG. 13J.

Further examples of information storage and reading are shown in FIGS. 11A-F. Images encoded in DNA, using a mixture of 1 message file and 1 noise file are shown in FIG. 11A. The top image corresponds to the message file (pre-hybridized to truth marker) and the bottom image corresponds to the noise file (pre-hybridized to false marker). The middle image corresponds to the recovered image after erasing the message by heating for 15 minutes at 95° C. The spatial distribution of missing pixels and incorrect pixels corresponding to noise are shown in FIG. 11B. The vertical gray stripe in the top image is expected because the first image has no encoded information there. The distribution of NGS reads across all pixels is shown in FIG. 11C. The distribution of the number of NGS reads matching perfectly in each pixel block is shown in FIG. 11D. In the second image, the “matched reads” correspond to the first image. The fraction of NGS reads mapped to each pixel block exactly matching the expected DNA message is shown in FIG. 11E. The fraction of each pixel block mapping to the highest frequency NGS read in each block (plurality) is shown in FIG. 11F.

Yet further examples of information storage and reading are shown in FIGS. 14A-F. Decoded images encoded in DNA, using a mixture of 1 message file and 7 noise files are shown in FIG. 14A. The message file was pre-hybridized to truth marker and noise files were pre-hybridized to false marker, respectively. Size of image was set as 240×320 upon decoding. The spatial distribution of missing pixels (black) and incorrect pixels corresponding to noise (gray) are sown in FIG. 14B. Outer parts of original image of the message file in 240×320 domain were displayed in gray. A pie chart showing the distribution of NGS reads is provided in FIG. 14C. The fraction of NGS reads match exactly to the original message file, the NGS reads match exactly to the original noise file, the ratio of NGS reads containing error in either address part or message part, and the ratio of NGS reads whose length is different from originally synthesized oligos are shown. The distribution of the number of exact NGS reads across all pixels is shown in FIG. 14D. Mapping the ratio of exact NGS reads mapping to each pixel is shown in FIG. 14E. The plurality ratio in each block, which corresponds to the fraction of each pixel block mapping to the number of dominant NGS reads, is shown in FIG. 14F.

An example of information storage and reading from a mixture of eight images is shown in FIGS. 12A-B. FIG. 12A shows the images after incubating the image mixture at 25° C. for 1 week. FIG. 12B shows the images after incubating the image mixture at 95° C. for 15 minutes.

An example of information storage and reading, showing information decay after 1 week, is provided in FIGS. 15A-F. Reading images encoded in DNA, using a mixture of 1 message file and 7 noise files are shown in FIG. 15A. Unlike FIGS. 14A-F, the mixture was incubated for 1 week at room temperature to test information decay, and then moved on to the next procedure for decoding/reading. Size of image was set as 240×320 upon decoding. The spatial distribution of missing pixels (black) and incorrect pixels corresponding to noise (gray) are shown in FIG. 15B. Outer parts of original image of the message file in 240×320 domain were displayed in gray. A pie chart showing the distribution of NGS reads is provided in FIG. 15C. The fraction of NGS reads match exactly to the original message file, the NGS reads match exactly to the original noise file, the ratio of NGS reads containing error in either address part or message part, and the ratio of NGS reads whose length is different from originally synthesized oligos are shown. Even after 1 week of incubation, the results hardly indicate information decay. The distribution of the number of exact NGS reads across all pixels are shown in FIG. 15D. Mapping the ratio of exact NGS reads mapping to each pixel is shown in FIG. 15E. The plurality ratio in each block, which corresponds to the fraction of each pixel block mapping to the number of dominant NGS reads, is shown in FIG. 15F.

An example of information erasure through heating the mixture at 95° C. for 15 minutes is shown in FIG. 16A-F. Reading images encoded in DNA, after erasing information in a mixture of 1 message file and 7 noise files are shown in FIG. 16A. All 8 images look alike, and it is hard to recognize the original image. Size of image was set as 240×320 upon decoding. FIG. 16B shows the spatial distribution of missing pixels (black) and incorrect pixels corresponding to noise (gray). Outer parts of original image of the message file in 240×320 domain were displayed in gray. After erasure, the majority of pixels correspond to noise. FIG. 16C provides a pie chart showing the distribution of NGS reads. The fraction of NGS reads match exactly to the original message file, the NGS reads match exactly to the original noise file, the ratio of NGS reads containing error in either address part or message part, and the ratio of NGS reads whose length is different from originally synthesized oligos are shown. After erasure, the perfect true message region became dominant, while the perfect noise/false message region decreased. The ratio of NGS reads whose length is different from originally synthesized oligos also increased. The distribution of the number of exact NGS reads across all pixels is shown in FIG. 16D. Although all 8 reading images look the same after erasure, some of the graphs have the patterns of the original images. It is because this graph is the result of matching the reading image to the original image. Mapping the ratio of exact NGS reads mapping to each pixel is shown in FIG. 16E. The plurality ratio in each block, which corresponds to the fraction of each pixel block mapping to the number of dominant NGS reads, is shown in FIG. 16F.

An example of incomplete information erasure through heating the mixture at 60° C. for 15 minutes is shown in FIGS. 17A-F. Reading images encoded in DNA, after erasing information in a mixture of 1 message file and 7 noise files are shown in FIG. 17A. Even with erasure at 60° C., original information (image) can be hardly recognized. Size of image was set as 240×320 upon decoding. The spatial distribution of missing pixels (black) and incorrect pixels corresponding to noise (gray) is shown in FIG. 17B. Outer parts of original image of the message file in 240×320 domain were displayed in gray. After erasure, the majority of pixels correspond to noise. FIG. 17C provides a pie chart showing the distribution of NGS reads. The fraction of NGS reads match exactly to the original message file, the NGS reads match exactly to the original noise file, the ratio of NGS reads containing error in either address part or message part, and the ratio of NGS reads whose length is different from originally synthesized oligos are shown. Compared to the file erased at 95° C., this file has slightly larger perfect true message region and smaller perfect noise/false message region. FIG. 17D provides the distribution of the number of exact NGS reads across all pixels. Although all 8 reading images look the same after erasure, some of the graphs have the patterns of the original images. It is because this graph is the result of matching the reading image to the original image. Mapping the ratio of exact NGS reads mapping to each pixel is shown in FIG. 17E. The plurality ratio in each block, which corresponds to the fraction of each pixel block mapping to the number of dominant NGS reads, is shown in FIG. 17F. In the histograms, the plurality ratio is distributed in a higher region than that of file erased at 95° C. (see FIG. 16F).

FIG. 18 provides a bar graph showing the ratio of correct, missing, and incorrect pixels of reading images. Ratios are the average values of 8 images. For original Twist pool, mixture of a message file and noise files, and the mixture incubated for 1 week at room temperature (Lanes 1-3) shows dominant ratio of correct pixels. On the other hand, in erased files (Lanes 4-6), incorrect or missing pixels are much dominant Lanes 5 and 6 are the graph analyzed with the reads whose plurality ratios are over 0.5. Truth markers and false markers are more distributed at 95° C. than 60° C., showing more missing pixels in files erased at 95° C.

### I. SYNTHESIS OF NUCLEIC ACIDS

The terms “nucleic acid molecule,” “nucleic acid polymer,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides (DNA) or ribonucleotides (RNA), or analogs thereof. A nucleic acid molecule is typically composed of a specific sequence of four nucleotide bases: adenine (A); cytosine (C); guanine (G); and thymine (T) (uracil (U) for thymine (T) when the polynucleotide is RNA). Thus, the term “nucleic acid sequence” is the alphabetical representation of a nucleic acid molecule. Nucleic acid molecules may optionally include one or more non-standard nucleotide(s), nucleotide analog(s) and/or modified nucleotides.

Any commercially available method of synthesizing nucleic acid molecules can be used. Nucleic acid molecules may be prepared using one or more of the phosphoramidite linkers and/or sequencing by ligation methods known to those of skill in the art. Oligonucleotide sequences may also be prepared by any suitable method, e.g., standard phosphoramidite methods such as those described herein below as well as those described by Beaucage and Carruthers ((1981) Tetrahedron Lett. 22: 1859) or the triester method according to Matteucci et al. (1981) J. Am. Chem. Soc. 103:3185), or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors.

These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A nucleic acid “region” or “domain” is a consecutive stretch of nucleotides of any length.

“Incorporating,” as used herein, means becoming part of a nucleic acid polymer.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

Examples of modified nucleotides include, but are not limited to diaminopurine, S2T, 5-fluorouracil, 5-bromouracil, 5-chlorouracil, 5-iodouracil, hypoxanthine, xantine, 4-acetylcyto sine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

An primer binding site may be added to a nucleic acid molecule during synthesis. For example, a primer binding site may be a sequence present in each truth marker DNA oligonucleotide in a population of truth marker DNA oligonucleotides. As such, when each truth marker DNA oligonucleotide is synthesized, a primer binding site is added to the 5′ end of the oligonucleotide.

### II. AMPLIFICATION OF NUCLEIC ACIDS

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. Primers may be no more than about 10, 12, 15, 20, 21, 22, 23, 24, 25, 26, 27, 28, 29, 30, 35, 40, 45, 50, 55, 60, 65, or 70 nucleotides in length.

The term “PCR” encompasses derivative forms of the reaction, including but not limited to, RT-PCR, real-time PCR, nested PCR, quantitative PCR, multiplexed PCR, assembly PCR and the like. Reaction volumes range from a few hundred nanoliters, e.g., 200 nL, to a few hundred microliters, e.g., 200 μL. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

Varied choices of polymerases exist with different properties, such as temperature, strand displacement, and proof-reading. Amplification can be isothermal, such as multiple displacement amplification (MDA) described by Dean et al., Comprehensive human genome amplification using multiple displacement amplification, Proc. Natl. Acad. Sci. U.S.A., vol. 99, p. 5261-5266. 2002; also Dean et al., Rapid amplification of plasmid and phage DNA using phi29 DNA polymerase and multiply-primed rolling circle amplification, Genome Res., vol. 11, p. 1095-1099. 2001; also Aviel-Ronen et al., Large fragment Bst DNA polymerase for whole genome amplification of DNA formalin-fixed paraffin-embedded tissues, BMC Genomics, vol. 7, p. 312. 2006. Amplification can also cycle through different temperature regiments, such as the traditional polymerase chain reaction (PCR) popularized by Mullis et al., Specific enzymatic amplification of DNA in vitro: The polymerase chain reaction. Cold Spring Harbor Symp. Quant. Biol., vole 51, p. 263-273. 1986. Other methods include Polony PCR described by Mitra and Church, In situ localized amplification and contact replication of many individual DNA molecules, Nuc. Acid. Res., vole 27, pages e 34. 1999; emulsion PCR (ePCR) described by Shendure et al., Accurate multiplex polony sequencing of an evolved bacterial genome, Science, vol. 309, p. 1728-32. 2005; and Williams et al., Amplification of complex gene libraries by emulsion PCR, Nat. Methods, vol. 3, p. 545-550. 2006. Any amplification method can be combined with a reverse transcription step, a priori, to allow amplification of RNA. According to certain aspects, amplification is not absolutely required since probes, reporters and detection systems with sufficient sensitivity can be used to allow detection of a single molecule using template non-hybridizing nucleic acid structures described. Ways to adapt sensitivity in a system include choices of excitation sources (e.g. illumination) and detection (e.g. photodetector, photomultipliers). Ways to adapt signal level include probes allowing stacking of reporters, and high intensity reporters (e.g. quantum dots) can also be used.

Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

A barcode, such as a sample barcode, may be added to the target nucleic acid molecules during amplification. One method involves annealing a primer (e.g., a truth marker DNA oligonucleotide) to the nucleic acid molecule, the primer including a first portion complementary to the nucleic acid molecule and a second portion including a barcode; and extending the annealed primer to form a barcoded nucleic acid molecule. Thus, the primer may include a 3′ portion and a 5′ portion, where the 3′ portion may anneal to a portion of the nucleic acid molecule and the 5′ portion comprises the barcode.

### III. SEQUENCING OF NUCLEIC ACIDS

Methods are also provided for the sequencing of the library of nucleic acid molecules. Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit, and additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). Additional methods for next-generation sequencing methods, including various methods for library construction that may be used with embodiments of the present disclosure are described, e.g., in Pareek (2011) and Thudi (2012).

In particular aspects, the sequencing technologies used in the methods of the present disclosure include the HiSeg™ system (e.g., HiSeg™ 2000 and HiSeg™ 1000) and the MiSeg™ system from Illumina, Inc. The HiSeg™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSeg™ system uses TruSeq™, Illumina's reversible terminator-based sequencing-by-synthesis.

Another example of a DNA sequencing platform is the QIAGEN GeneReader platform—a next generation sequencing (NGS) platform utilizing proprietary modified nucleotides whose 3′ OH groups are reversely terminated by a small moiety to perform sequencing-by-synthesis (SBS) in a massively parallel manner Briefly, the sequencing templates are first clonally amplified on a solid surface (such as beads) to generate hundreds of thousands of identical copies for each individual sequencing template, denaturized to generate single-stranded sequencing templates, hybridized with sequencing primer, and then immobilized on the flow cell. The immobilized sequencing templates are then subjected to a nucleotide incorporation reaction in a reaction mix that includes modified nucleotides with a cleavable 3′ blocking group that enables the incorporation and detection of only one specific nucleotide onto each sequencing template in each cycle. See U.S. Pat. Nos. 6,664,079; 8,612,161; and 8,623,598, each of which is incorporated by reference herein.

Another example of a DNA sequencing platform is the Ion Torrent PGM™ sequencer (Thermo Fisher) and the Ion Torrent Proton™ Sequencer (Thermo Fisher), which are ion-based sequencing systems that sequence nucleic acid templates by detecting ions produced as a byproduct of nucleotide incorporation. Typically, hydrogen ions are released as byproducts of nucleotide incorporations occurring during template-dependent nucleic acid synthesis by a polymerase. The Ion Torrent PGM™ sequencer and Ion Proton™ Sequencer detect the nucleotide incorporations by detecting the hydrogen ion byproducts of the nucleotide incorporations. The Ion Torrent PGM™ sequencer and Ion Torrent Proton™ sequencer include a plurality of nucleic acid templates to be sequenced, each template disposed within a respective sequencing reaction well in an array. The wells of the array are each coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor provides output signals indicative of nucleotide incorporation, which can be represented as voltage changes whose magnitude correlates with the H+ ion concentration in a respective well or reaction chamber. Different nucleotide types are flowed serially into the reaction chamber, and are incorporated by the polymerase into an extending primer (or polymerization site) in an order determined by the sequence of the template. Each nucleotide incorporation is accompanied by the release of H+ ions in the reaction well, along with a concomitant change in the localized pH. The release of H+ ions is registered by the FET of the sensor, which produces signals indicating the occurrence of the nucleotide incorporation. Nucleotides that are not incorporated during a particular nucleotide flow will not produce signals. The amplitude of the signals from the FET may also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers permit the instrument to resolve the sequence of many nucleic acid templates simultaneously. Further details regarding the compositions, design and operation of the Ion Torrent PGM™ sequencer can be found, for example, in U.S. Pat. Publn. Nos. 2009/0026082; 2010/0137143; and 2010/0282617, all of which are incorporated by reference herein in their entireties.

Another example of a DNA sequencing technique that can be used in the methods of the present disclosure is 454 sequencing (Roche) (Margulies et al., 2005). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2010). Complete genomics' CGA Platform uses a novel strategy called combinatorial probe anchor ligation (cPAL) for sequencing. The process begins by hybridization between an anchor molecule and one of the unique adapters. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n+1, n+2, n+3, and n+4 positions.

A further sequencing platform includes nanopore sequencing (Oxford Nanopore). Nanopore detection arrays are described in US2011/0177498; US2011/0229877; US2012/0133354; WO2012/042226; WO2012/107778, and have been used for nucleic acid sequencing as described in US2012/0058468; US2012/0064599; US2012/0322679 and WO2012/164270, all of which are hereby incorporated by reference. A single molecule of DNA can be sequenced directly using a nanopore, without the need for an intervening PCR amplification step or a chemical labelling step or the need for optical instrumentation to identify the chemical label. Commercially available nanopore nucleic acid sequencing units are developed by Oxford Nanopore (Oxford, United Kingdom). The GridION™ system and miniaturised MinION™ device are designed to provide novel qualities in molecular sensing such as real-time data streaming, improved simplicity, efficiency and scalability of workflows and direct analysis of the molecule of interest. Using the Oxford Nanopore nanopore sequencing platform, an ionic current is passed through the nanopore by setting a voltage across this membrane. If an analyte passes through the pore or near its aperture, this event creates a characteristic disruption in current. Measurement of that current makes it possible to identify the molecule in question. For example, this system can be used to distinguish between the four standard DNA bases G, A, T and C, and also modified bases. It can be used to identify target proteins, small molecules, or to gain rich molecular information, for example to distinguish between the enantiomers of ibuprofen or study molecular binding dynamics. These nanopore arrays are useful for scientific applications specific for each analyte type; for example when sequencing DNA, the technology may be used for resequencing, de novo sequencing, and epigenetics.

### IV. KITS

The technology herein includes kits for creating libraries of nucleic acids molecules for storing information. A “kit” refers to a combination of physical elements. For example, a kit may include, for example, one or more components, such as specific primers, enzymes, reaction buffers, an instruction sheet, and other elements useful to practice the technology described herein. These physical elements can be arranged in any way suitable for carrying out the disclosure.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present disclosure also will typically include a means for containing the nucleic acids, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the disclosure. Such kits, however, are not limited to the particular items identified above.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Storing Information in Nucleic Acid Molecules & Erasing and Reading Information Stored Therein

Selectively amplifying DNA from oligo pool. A chip-synthesized DNA oligonucleotide pool was ordered from TWIST Bioscience, containing a total of 93,894 DNA oligonucleotides encoding eight separate bitmap image files. All oligonucleotides were 120 nucleotides long. After receiving the pool in dry (lyophilized) form, lx Tris-EDTA buffer was added such that the total concentration would be 10 ng/μL. Then, the pool was diluted 10,000-fold using MilliQ water with 0.1% Tween-20 to form a secondary stock.

Primers for amplifying different subpools of oligos (corresponding to the eight separate bitmap image files) were ordered from Integrated DNA Technologies. The forward primers were phosphorylated on their 5′ ends. The reverse primers have three phosphorothioated DNA bases on their 5′ ends.

5 μL of the oligo pool secondary stock was mixed with 5 μL of the forward primer (4 μM), 5 μL of the reverse primer (4 μM) reverse primer, 25 μL KAPA Hifi enzyme mix, and 10 μL MilliQ water in a 0.6 mL Eppendorf tube. This 50 μL mix was then amplified via PCR using the following thermocycling protocol: (1) 95° C. for 3 mM, (2) 98° C. for 20 sec, (3) 60° C. for 15 sec, (4) 72° C. for 15 sec, (5) repeat (2)-(4) for 32 times, (6) 72° C. for 1 mM (33 cycles of amplification in total). The 50 μL amplicon solution was then purified using Agencourt AMPure XP beads (90 μL, 1.8×) following manufacturer specifications.

Subsequently, 20 μL of the purified amplicon solution was mixed with 1 μL Lambda Exonuclease enzyme (New England Biolabs), 3 μL Lambda Exonuclease reaction buffer (10×), and 6 μL MilliQ water. The mixture was incubated at 37° C. for 30 minutes and then at 75° C. for 10 minutes, in order to digest phosphorylated DNA molecules (extended forward primers), but not the phosphorothioated DNA molecules (extended reverse primers). The products of this reactions were then purified using an Oligo Clean & Concentrator kit (Zymo Research) according to manufacturer specifications. The purified products were then quantitated using a Qubit ssDNA Assay kit.

To purify amplicons of DNA subpools intended to be information DNA molecules (examples of which are provided in Table 1), 0.5 x relative amount of truth marker oligonucleotides were added. To purify amplicons of DNA subpools intended to be obfuscation DNA molecules (examples of which are provided in Table 2), 1.5 x relative amount of false marker oligonucleotides were added. The solutions were individually thermally annealed, and then mixed at room temperature to form the DNA solution with erasable information.

Information erasing protocol. The mixture of information DNA molecules and obfuscation DNA molecules were heated to 95° C. for 15 min and then cooled down to the room temperature.

Information reading protocol. To 4 μL of the mixture of information DNA molecules and obfuscation DNA molecules, 2 μL of Klenow fragment DNA polymerase, 1 mM dNTP mixture, 2 μL NEB Buffer 2, and 10.75 μL MilliQ water were added. The mixture was then incubated at 37° C. for 1 hour to extend the truth markers.

Subsequently, the sample was diluted 10 x using MilliQ water with 0.1% Tween-20. To 2.5 μL of the diluted mix, 12.5 μL KAPA Hifi enzyme mix, 2.5 μL forward primer (4 μM), 5 μL reverse primer mixture (4 μM), and 2.5 μL MilliQ water were added. This 25 μL mix was amplified via PCR using the following thermocycle profile: (1) 95° C. for 3 min, (2) 98° C. for 20 sec, (3) 60° C. for 15 sec, (4) 72° C. for 15 sec, (5) repeat (2)-(4) once, (6) 72° C. for 1 min (2 cycles of amplification in total).

Preparation for NGS. Index primers were appended using the Nextera XT kit and the KAPA Hifi enzyme mix following manufacturer specifications. Amplicons were purified using Agencourt AMPure XP beads, and then quantitated using a Qubit dsDNA HS Assay kit and diluted to the recommended concentration suggested by Illumina for the MiSeq instrument. Purified amplicons were also subject to a quality control assay using a Bioanalyzer capillary electrophoresis assay (Agilent). PhiX DNA solution was spiked in to occupy 20% of all molecules, consistent with Illumina recommendations. This final library was then run on an Illumina Miseq instrument using a v3-150 cycle kit.

TABLE 1
Example DNA Sequences used for information
DNA molecules

CGAAAGCCTGCAGAACGTTTATTTAAGTGCAGTGCACCTCGAGTCA
GTGGAGACGTCTCGCTACGAGGTCGACACACCTCCTTGGTCTGGAG
TCGCAATCGTAACCATAGCAATCCAAAC
(SEQ ID NO: 1)
CGAAAGCCTGCAGAACGTTTATTTATCTGCAGTGCAGCTCGAGTCC
ACTCTCTCGCAAGGGTTCGCACTCCTGTCTCTGGCTTCGAGTCGGA
ACGCAATCGTAACCATAGCAATCCAAAC
(SEQ ID NO: 2)
CGAAAGCCTGCAGAACGTTTATTTAACTGCAGTGCAGCTCTCGTCC
AGTCTGCAGAGGAGGAGAGCTGTCAGGTCGTGTCTGGAGTCACGCT
ACGCAATCGTAACCATAGCAATCCAAAC
(SEQ ID NO: 3)
CGAAAGCCTGCAGAACGTTTATTTAGATGCAGTGCAGTGGACCTCG
ACTCGTCAGTGCAGAGCAGCACTCCTGTCTGCTCCTGAGAGGAGTC
GAGCAATCGTAACCATAGCAATCCAAAC
(SEQ ID NO: 4)
CGAAAGCCTGCAGAACGTTTATTTACATGCAGTGCCTTCCACTCCT
GACCGTAGGTCAGGCTAGGCAGACTGGACTCGACACACGGTTCGTG
ACGCAATCGTAACCATAGCAATCCAAAC
(SEQ ID NO: 5)

TABLE 2
Example DNA Sequences used for obfuscation DNA molecules
CGAAAGCCTGCAGAACGTTTATTTAAGTGCAGTGCCAACTGTACTTCGATGAACTCAA
CTAGGATACACTACGATACGATAGACTAGGATAGGATCAAAGCATAGCAAAGGAATG
GAATG
(SEQ ID NO: 6)
CGAAAGCCTGCAGAACGTTTATTTATCTGCAGTGCCTACTCTTCTTCGATGTACTGTT
CTAGGATTGGATTGACTTCGATTGGATTGACTTCGATCAAAGCATAGCAAAGGAATG
GAATG
(SEQ ID NO: 7)
CGAAAGCCTGCAGAACGTTTATTTAACTGCAGTGCTGACTGTTCTTCGATAGACTCTT
CTACGATGAACTCATCTTCGATGAACTCATCTTCGATCAAAGCATAGCAAAGGAATGG
AATG
(SEQ ID NO: 8)
CGAAAGCCTGCAGAACGTTTATTTAGATGCAGTGCAGACTACACTACGATCAACTCTA
CTCTGATCTTCTTGACTTCGATTCACTACACTCAGATCAAAGCATAGCAAAGGAATGG
AATG
(SEQ ID NO: 9)
CGAAAGCCTGCAGAACGTTTATTTACATGCAGTGCGAACTTGACTACGATCTACTACA
CTGTGATGAACTTCACTGTGATCTACTCAACTAGCATCAAAGCATAGCAAAGGAATGG
AATG
(SEQ ID NO: 10)
CGAAAGCCTGCAGAACGTTTATTTAAGTGCAGTGCCTCTTCTCTTCTCTTCTCTTCTCT
TCTCTTCTCTTCTCTTCTCTTCTCTTCTCTTCTCTTCAAAGGAAACGATTCCAAACGAA
AC
(SEQ ID NO: 11)
CGAAAGCCTGCAGAACGTTTATTTATCTGCAGTGCCTCTTCTCTTCTCTTCTCTTCTCT
TCTCTTCTCTTCTCTTCTCTTCTCTTCTCTTCTCTTCAAAGGAAACGATTCCAAACGAA
AC
(SEQ ID NO: 12)
CGAAAGCCTGCAGAACGTTTATTTAACTGCAGTGCCTCTTCTCTTCTCTTCTCTTCTCT
TCTCTTCTCTTCTCTTCTCTTCTCTTCTCTTCTCTTCAAAGGAAACGATTCCAAACGAA
AC
(SEQ ID NO: 13)
CGAAAGCCTGCAGAACGTTTATTTAGATGCAGTGCCTCTTCTCTTCTCTTCTCTTCTCT
TCTCTTCTCTTCTCTTCTCTTCTCTTCTCTTCTCTTCAAAGGAAACGATTCCAAACGAA
AC
(SEQ ID NO: 14)
CGAAAGCCTGCAGAACGTTTATTTACATGCAGTGCCTCTTCTCTTCTCTTCTCTTCTCT
TCTCTTCTCTTCTCTTCTCTTCTCTTCTCTTCTCTTCAAAGGAAACGATTCCAAACGAA
AC
(SEQ ID NO: 15)
CGAAAGCCTGCAGAACGTTTATTTAAGTGCAGTGCTGCTAAGCTACTTGTGACTATGC
TAGATGTTCCTATCCTATGAGTTGAGTGATGTTGTCTCATAGCAAAGGTATGCAAAGG
AAAG
(SEQ ID NO: 16)
CGAAAGCCTGCAGAACGTTTATTTATCTGCAGTGCTGGTAGACTATGAGTAGGTAGT
CAAGTCTATGCTAGTCTAACAGTTCGTACACAAGACTACATAGCAAAGGTATGCAAAG
GAAAG
(SEQ ID NO: 17)
CGAAAGCCTGCAGAACGTTTATTTAACTGCAGTGCACGAAGTGTAACTGTTCGTAGT
GTATGAGTACGAAACGTATGTGTACGTAACGTACATGTCATAGCAAAGGTATGCAAA
GGAAAG
(SEQ ID NO: 18)
CGAAAGCCTGCAGAACGTTTATTTAGATGCAGTGCAGCAAAGTGTCAAGTAGTGTCT
AGTAGGATTCCAATGTGTGAAGTCTGTAAGTGTACTCTCATAGCAAAGGTATGCAAA
GGAAAG
(SEQ ID NO: 19)
CGAAAGCCTGCAGAACGTTTATTTACATGCAGTGCCAAGTGAAGTTGTCTAGAGTAG
AGTCTAGTGTAGTTCAGTCTAGTCAAGTCAAGTACTCTCATAGCAAAGGTATGCAAAG
GAAAG
(SEQ ID NO: 20)

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 9,384,320
U.S. Pat. No. 9,774,351
U.S. Pat. Appln. Publn. No. 2017/0017436
U.S. Pat. Appln. Publn. No. 2015/0261664
European Pat. Appln. Publn. No. 2947589A1
European Pat. Appln. Publn. No. 3173961A1
PCT Appln. Publn. No. WO2016/023784
PCT Appln. Publn. No. WO2017/153351

What is claimed is:

1. A composition comprising a population of DNA molecules, the population comprising true information DNA molecules, false obfuscation DNA molecules, and truth marker DNA oligonucleotides,
wherein the true information DNA molecules and the false obfuscation DNA molecules each comprise a first sequence that is complementary to a portion of a sequence of the truth marker DNA oligonucleotides,
wherein the first sequence of the true information DNA molecules is hybridized to the truth marker DNA oligonucleotides,
wherein the first sequence of the false obfuscation DNA molecules is not hybridized to the truth marker DNA oligonucleotides,
wherein the true information DNA molecules and the false obfuscation DNA molecules each comprise an address region,
wherein the address region of each true information DNA molecule is unique among the true information DNA molecules in the population,
wherein one true information DNA molecule and at least one false information DNA molecule in the population share an identical address region.

2. The composition of claim 1, wherein the first sequence of the false obfuscation DNA molecules is single stranded.

3. The composition of claim 1, wherein the population further comprises false marker DNA oligonucleotides.

4. The composition of claim 3, wherein a portion of the false marker DNA oligonucleotides is at least partially complementary to the first sequence of both the true information DNA molecules and the false obfuscation DNA molecules.

5. The composition of claim 3, wherein the false marker DNA oligonucleotides and the truth marker DNA oligonucleotides comprise different sequences.

6. The composition of any one of the claims 3-5, wherein the false marker DNA oligonucleotides comprise a chemical functionalization.

7. The composition of any one of claims 3-6, wherein the first sequence of the false obfuscation DNA molecules is hybridized to the false marker DNA oligonucleotides.

8. The composition of any one of claims 3-7, wherein the false marker DNA oligonucleotides comprise a 3′ functionalization that prevents extension by a DNA polymerase.

9. The composition of any one of claims 1-8, wherein the first sequence is between 10 and 50 nucleotides long.

10. The composition of any one of claims 1-9, wherein the true information DNA molecules and the false obfuscation DNA molecules are each, independently, between 50 and 2000 nucleotides long.

11. The composition of any one of claims 1-10, wherein the first regions of the true information DNA molecules are located towards the 5′ end of the true information DNA molecules.

12. The composition of any one of claims 1-11, wherein the truth marker DNA oligonucleotides comprise a primer binding region that is not complementary to the true information DNA molecules.

13. A method of encoding an information-bearing file or an obfuscation file in information DNA molecules, the method comprising:
(a) obtaining an input file in ASCII/hexadecimal format;
(b) independently translating each ASCII character/byte from 00 to FF in hexadecimal to a five nucleotide DNA sequence;
(c) dividing the concatenated DNA sequence representing the entire input file into a set of message sequences;
(d) providing and encoding in DNA a unique address sequence identifying the position within the DNA sequence for each message sequence;
(e) designing a truth marker binding region sequence;
(f) constructing information DNA molecule sequences by concatenating from 5′ to 3′ the truth marker binding region sequence, the unique address sequences, and corresponding message sequences; and
(g) chemically synthesizing information DNA molecules comprising the information DNA molecule sequences.

14. The method of claim 13, wherein the information-bearing DNA molecules further comprises one or more primer binding regions located on the 5′ and/or 3′ end of the information DNA molecule sequence.

15. The method of claim 13, wherein the obfuscation DNA molecules further comprises one or more primer binding regions located on the 5′ and/or 3′ end of the information DNA molecule sequence.

16. The method of claim 13, wherein step (b) comprises converting each hexadecimal character to its binary, 8 bit representation and then converting each binary, 8 bit representation to one 2-bit region and two 3-bit regions, wherein the 2-bit region is mapped to G, C, A, or T, and wherein the 3-bit regions are each mapped to CA, CT, GA, GT, TC, TG, AC, or AG.

17. A population of information DNA molecules made by the method of any one of claims 13-16.

18. A method of preparing a DNA solution encoding information that is amenable to rapid erasure, the method comprising:
(a) obtaining a solution of information DNA molecules encoding an information-bearing file prepared according to the method of any one of claims 13-17;
(b) hybridizing the solution of information DNA molecules to a solution of truth marker DNA oligonucleotide molecules;
(c) obtaining at least one solution of obfuscation DNA molecules encoding an obfuscation file prepared according to the method of any one of claims 13-17; and
(d) combining the hybridized solution of part (b) with the at least one solution of obfuscation DNA molecules of part (c).

19. The method of claim 18, further comprising hybridizing the at least one solution of obfuscation DNA molecules to a solution of false marker DNA oligonucleotide molecules prior to combining in part (d).

20. The method of claim 18 or 19, wherein the truth marker DNA oligonucleotides are present at a molar quantity that is smaller than or equal to the molar quantity of information DNA molecules.

21. The method of claim 19, wherein the false marker DNA oligonucleotides are present at a molar quantity that is greater than or equal to the molar quantity of obfuscation DNA molecules.

22. The method of any one of claims 18-21, wherein the hybridizing of part (b) comprises heating the combined solutions to at least 70° C. and then cooling the combined solutions to 50° C. or lower.

23. The method of any one of claims 19-22, wherein hybridizing the at least one solution of obfuscation DNA molecules to a solution of false marker DNA oligonucleotide molecules prior to combining in part (d) comprises heating the combined solutions to at least 70° C. and then cooling the combined solutions to 50° C. or lower.

24. A DNA solution encoding information that is amenable to rapid erasure made by the method of any one of claims 18-23.

25. A method of erasing information encoded in a DNA solution of any one of claims 1-12, the method comprising heating the DNA solution an elevated temperature for a duration of no less than 15 seconds.

26. The method of claim 25, wherein the elevated temperature is approximately 50° C., 55° C., 60° C., 65° C., 70° C., 75° C., 80° C., 85° C., 90° C., 95° C., or 100° C.

27. The method of claim 25 or 26, wherein the duration of the heating is approximately 15 seconds, 30 seconds, 45 seconds, 1 minute, 2 minutes, 3 minutes, 5 minutes, 10 minutes, 15 minutes, 20 minutes, 30 minutes, or 60 minutes.

28. A method of reading information encoded in a DNA solution of any one of claims 1-12, the method comprising:
(a) adding a DNA polymerase, dNTPs, and buffers to the solution;
(b) incubating the mixture of part (a) at a temperature amenable to enzymatic extension of the truth marker based on the hybridized information DNA molecules;
(c) preparing a next-generation sequencing (NGS) library based on the polymerase-extended truth markers of part (b);
(d) performing NGS;
(e) analyzing NGS reads to determine the dominant message sequence for each address sequence; and
(f) reassembling the information-bearing file from the dominant message sequence for each address sequence.

29. The method of claim 28, wherein the preparation of the NGS library based on polymerase-extended truth markers comprises ligation of sequencing adaptors to double-stranded DNA molecules.

30. The method of claim 29, wherein the NGS library preparation further comprises polymerase chain reaction (PCR) amplification using sequencing adaptors.

31. The method of claim 28, wherein the preparation of the NGS library based on polymerase-extended truth markers comprises polymerase chain reaction (PCR) amplification comprising a primer that includes a sequencing adaptor at or near the 5′ region and a sequence specific to the truth marker DNA oligonucleotide but not to the false marker DNA oligonucleotide.

32. The method of any one of claims 29-31, wherein the NGS library preparation further comprises appending sample indexes using PCR.

33. A method of erasing information encoded in a DNA solution of any one of claims 1-12, the method comprising exposing the DNA solution to a temperature above room temperature for a duration of no less than the estimated half-life of the duplex comprising the truth marker oligonucleotide and the first sequence.

34. The method of claim 33, where the half-life is calculated as
$${t}_{1 / 2} = \frac{{e}^{{ΔG}^{\underline{o}} / RT}}{{k}_{f}}$$
where t_{1/2} is half-life, R is the gas constant, T is the exposure temperature, ΔG° is the Gibbs free energy hybridization of the duplex, and k_{f}(=10⁶ M·⁻¹ s⁻¹) is the rate constant of hybridization.


SEQUENCE LISTING (21 sequences)

SEQ ID NO: 1 - DNA, 120 bp, Artificial Sequence
  Artificial polynucleotide
cgaaagcctgcagaacgtttatttaagtgcagtgcacctcgagtcagtggagacgtctcgctacgaggtcgacacacctccttggtctggagtcgcaatcgtaaccatagcaatccaaac

SEQ ID NO: 2 - DNA, 120 bp, Artificial Sequence
  Artificial polynucleotide
cgaaagcctgcagaacgtttatttatctgcagtgcagctcgagtccactctctcgcaagggttcgcactcctgtctctggcttcgagtcggaacgcaatcgtaaccatagcaatccaaac

SEQ ID NO: 3 - DNA, 120 bp, Artificial Sequence
  Artificial polynucleotide
cgaaagcctgcagaacgtttatttaactgcagtgcagctctcgtccagtctgcagaggaggagagctgtcaggtcgtgtctggagtcacgctacgcaatcgtaaccatagcaatccaaac

SEQ ID NO: 4 - DNA, 120 bp, Artificial Sequence
  Artificial polynucleotide
cgaaagcctgcagaacgtttatttagatgcagtgcagtggacctcgactcgtcagtgcagagcagcactcctgtctgctcctgagaggagtcgagcaatcgtaaccatagcaatccaaac

SEQ ID NO: 5 - DNA, 120 bp, Artificial Sequence
  Artificial polynucleotide
cgaaagcctgcagaacgtttatttacatgcagtgccttccactcctgaccgtaggtcaggctaggcagactggactcgacacacggttcgtgacgcaatcgtaaccatagcaatccaaac

SEQ ID NO: 6 - DNA, 120 bp, Artificial Sequence
  Artificial polynucleotide
cgaaagcctgcagaacgtttatttaagtgcagtgccaactgtacttcgatgaactcaactaggatacactacgatacgatagactaggataggatcaaagcatagcaaaggaatggaatg

SEQ ID NO: 7 - DNA, 120 bp, Artificial Sequence
  Artificial polynucleotide
cgaaagcctgcagaacgtttatttatctgcagtgcctactcttcttcgatgtactgttctaggattggattgacttcgattggattgacttcgatcaaagcatagcaaaggaatggaatg

SEQ ID NO: 8 - DNA, 120 bp, Artificial Sequence
  Artificial polynucleotide
cgaaagcctgcagaacgtttatttaactgcagtgctgactgttcttcgatagactcttctacgatgaactcatcttcgatgaactcatcttcgatcaaagcatagcaaaggaatggaatg

SEQ ID NO: 9 - DNA, 120 bp, Artificial Sequence
  Artificial polynucleotide
cgaaagcctgcagaacgtttatttagatgcagtgcagactacactacgatcaactctactctgatcttcttgacttcgattcactacactcagatcaaagcatagcaaaggaatggaatg

SEQ ID NO: 10 - DNA, 120 bp, Artificial Sequence
  Artificial polynucleotide
cgaaagcctgcagaacgtttatttacatgcagtgcgaacttgactacgatctactacactgtgatgaacttcactgtgatctactcaactagcatcaaagcatagcaaaggaatggaatg

SEQ ID NO: 11 - DNA, 120 bp, Artificial Sequence
  Artificial polynucleotide
cgaaagcctgcagaacgtttatttaagtgcagtgcctcttctcttctcttctcttctcttctcttctcttctcttctcttctcttctcttctcttcaaaggaaacgattccaaacgaaac

SEQ ID NO: 12 - DNA, 120 bp, Artificial Sequence
  Artificial polynucleotide
cgaaagcctgcagaacgtttatttatctgcagtgcctcttctcttctcttctcttctcttctcttctcttctcttctcttctcttctcttctcttcaaaggaaacgattccaaacgaaac

SEQ ID NO: 13 - DNA, 120 bp, Artificial Sequence
  Artificial polynucleotide
cgaaagcctgcagaacgtttatttaactgcagtgcctcttctcttctcttctcttctcttctcttctcttctcttctcttctcttctcttctcttcaaaggaaacgattccaaacgaaac

SEQ ID NO: 14 - DNA, 120 bp, Artificial Sequence
  Artificial polynucleotide
cgaaagcctgcagaacgtttatttagatgcagtgcctcttctcttctcttctcttctcttctcttctcttctcttctcttctcttctcttctcttcaaaggaaacgattccaaacgaaac

SEQ ID NO: 15 - DNA, 120 bp, Artificial Sequence
  Artificial polynucleotide
cgaaagcctgcagaacgtttatttacatgcagtgcctcttctcttctcttctcttctcttctcttctcttctcttctcttctcttctcttctcttcaaaggaaacgattccaaacgaaac

SEQ ID NO: 16 - DNA, 120 bp, Artificial Sequence
  Artificial polynucleotide
cgaaagcctgcagaacgtttatttaagtgcagtgctgctaagctacttgtgactatgctagatgttcctatcctatgagttgagtgatgttgtctcatagcaaaggtatgcaaaggaaag

SEQ ID NO: 17 - DNA, 120 bp, Artificial Sequence
  Artificial polynucleotide
cgaaagcctgcagaacgtttatttatctgcagtgctggtagactatgagtaggtagtcaagtctatgctagtctaacagttcgtacacaagactacatagcaaaggtatgcaaaggaaag

SEQ ID NO: 18 - DNA, 120 bp, Artificial Sequence
  Artificial polynucleotide
cgaaagcctgcagaacgtttatttaactgcagtgcacgaagtgtaactgttcgtagtgtatgagtacgaaacgtatgtgtacgtaacgtacatgtcatagcaaaggtatgcaaaggaaag

SEQ ID NO: 19 - DNA, 120 bp, Artificial Sequence
  Artificial polynucleotide
cgaaagcctgcagaacgtttatttagatgcagtgcagcaaagtgtcaagtagtgtctagtaggattccaatgtgtgaagtctgtaagtgtactctcatagcaaaggtatgcaaaggaaag

SEQ ID NO: 20 - DNA, 120 bp, Artificial Sequence
  Artificial polynucleotide
cgaaagcctgcagaacgtttatttacatgcagtgccaagtgaagttgtctagagtagagtctagtgtagttcagtctagtcaagtcaagtactctcatagcaaaggtatgcaaaggaaag

SEQ ID NO: 21 - DNA, 20 bp, Artificial Sequence
  Artificial polynucleotide
cgaactagaattcccagatc